(19) United States
(12) Patent Application Publication
(10) Pub. No.: US 2016/0296111 A1
(43) Pub. Date: Oct. 13, 2016

(54) Optical Accessory For A Mobile Device

(71) Applicant: D-EYE S.r.l., Padova (IT)

(72) Inventor: Andrea RUSSO, Brescia (IT)

(21) Appl. No.: 15/036,765

(22) PCT Filed: Apr. 18, 2014
(86) PCT No.: PCT/IB2014/060842

§ 371 Date: May 13, 2016

(30) Foreign Application Priority Data
Nov. 15, 2013 (IT) BS2013A000169

(51) Int. Cl. A61B 3/12; G03B 17/56; A61B 3/14

(52) U.S. Cl. CPC A61B 3/12; A61B 3/14; G03B 17/565

(57) ABSTRACT

An optical accessory, which can be associated with a mobile device such as a smartphone or tablet, is described. The accessory comprises coupling means which allow it to be firmly positioned in correspondence with both a camera lens and a light source of the mobile device. The optical accessory further comprises a first optical element provided with a first reflective surface, for example a mirror. The mirror surface reflects a source light beam emitted by the light source and generates a first reflected beam directed toward the visual field of the camera lens. Moreover, the accessory comprises a second optical element, i.e. a beam splitter, provided with a partially reflective second surface, generating a second reflected beam directed to illuminate the eye of a patient and substantially inside the visual field of the camera lens so as to realize an ophthalmoscopic system which allows the examination of the “fundus oculi” of the eye.

### FIELD OF THE INVENTION

The present invention is part of the field of optical instruments used in particular, but not exclusively, in the medical industry and specifically relates to an optical accessory applied to a mobile device provided with a camera, in particular a smartphone or tablet, to realize an ophthalmoscope.

### STATE OF THE ART

As known, the ophthalmoscope is an optical device allowing a doctor to view the eyeground or “fundus oculi” of a human eye, in order to diagnose ocular diseases (glaucoma, macular degeneration, venous and arterial occlusions, etc.) and/or systemic diseases (diabetes, arterial hypertension, intracranial hypertension, etc.).

Nowadays, this examination is carried out in eye care clinics by means of non-portable ophthalmoscopic instruments positioned on bulky and expensive supporting structures which implement both optical and computer equipments assisting the optical equipment. However, this type of instruments has proved to be impractical and greatly limiting the observation of the “fundus oculi”, in particular in bedridden, paediatric, disabled patients, or in any case patients who cannot immediately access an eye care clinic.

To overcome the aforesaid drawbacks, portable ophthalmoscopes have been developed, as described for example in US2013128223.

These instruments mainly comprise a lighting system having at least a light source for illuminating the eye of a patient. A binocular vision system used by the doctor and associated to the lighting system allows to view the eye as illuminated by the light source.

With the recent coming of smartphones and tablets provided with cameras, the portable ophthalmoscope was associated to these mobile devices by positioning a detachable support. In particular, the support is adapted to house the mobile device such that the camera can be positioned at the observing portion of the ophthalmoscope, i.e. the portion intended for allowing the user to carry out the observation. In this way, the camera acts as a digital-image acquisition device; however, the light source is still provided by the instrument.

Such a system can be seen, for example, in the website www.welchallyn.com/promotions/iExaminer/index.html.

Diffusion and the usability of this system are limited because it is very expensive, being further bulky and not very ergonomic.

In addition, the application of the mobile device only as an image acquisition device functionally restricts its use, in particular as regards the extension of the visual field that can be obtained and, therefore, the accuracy of the examination.

In addition, for unskilled personnel is very difficult to use this system. In particular, the support of the mobile device can be misaligned with respect to the instrument, thereby requiring frequent adjustments of the position.

### SUMMARY OF THE INVENTION

It is therefore an object of the present invention to provide an optical accessory for a mobile device provided with a camera which is able to solve the problems of the known art systems.

It is another object of the present invention to provide an optical accessory smaller than known devices.

It is a further object of the present invention to provide an optical accessory for a mobile device of simple structure and being cost effective.

These and other objects are achieved by an optical accessory for a mobile device according to claim 1. Mobile device means a smartphone, a tablet or another similar electronic device provided with a camera, as described below.

In particular, the optical accessory comprises coupling means to be functionally attached to the mobile device thereby substantially forming an ophthalmoscope, as described below.

By the coupling means the optical accessory can be removably attached in correspondence with a camera lens and a respective light source provided by the mobile device. Camera lens means a lens able to capture digital images in form of photographs or videos.

The optical accessory further comprises a first optical element provided with a first reflective surface, in particular a mirror, apt to reflect a source light beam emitted by the light source and to generate a first reflected beam directed toward the visual field of the camera lens. In other words, the mirror only reflects, or deviates, the source light beam in the visual field of the camera lens.

In addition, the accessory comprises a second optical element, in particular a beam splitter, comprising a partially reflective second surface, preferably parallel to the first reflective surface. The second surface, on which the first reflected beam is incident, generates a second reflected beam directed outside the accessory toward an eye to be examined. The second reflected beam is substantially inside the visual field of the camera lens, i.e. substantially aligned with it to allow the view of the “fundus oculi” of the eye itself.

In particular, by positioning the optical accessory applied to the mobile device close to the eye to be examined so that the second light beam is incident on the eye, the eye can be viewed and its digital images can be captured, through the camera lens.

In particular, the first surface is a flat surface and is inclined at an angle α comprised between 30° and 60° with respect to a lying plane P of the said camera lens and said light source. Also the second surface is a flat surface inclined at an angle α′ comprised between 30° and 60° with respect to the lying plane P.

Preferably, the first surface is inclined at about 45° with respect to the plane P and the second surface is parallel to the first surface and also inclined at 45°. In this way, the source light beam emitted by the light source, generally a LED source, is reflected by 90° toward the visual field in front of the camera lens.

Preferably, the first surface is inclined at about 52.5° with respect to the plane P and the second surface is parallel to the first surface and also inclined at 52.5°.

In this way, the source light beam emitted by the light source, generally a LED source, is reflected by 97.5° toward the visual field in front of the camera lens. In front of the camera lens the beam splitter is positioned, having the partially reflective surface parallel to the first surface of the mirror. The first reflected beam is incident on the second surface of the beam splitter and generates, respectively, a transmitted beam passing through the beam splitter and a second reflected beam directed outside of the accessory for illuminating an eye to be examined.

The second reflected beam is substantially inside the visual field of the camera lens and, once positioned in-line with the eye, allows to carry out visual examination of the “fundus oculi” and its image to be captured by the lens.

Since the above mentioned system uses both the camera lens and the light source of the mobile device, i.e. of the smartphone, it is a small accessory easy to be used even by unskilled personnel.

Preferably, a light-absorbing wall is provided which is associated to the second optical element and placed opposite to the first optical element. The light-absorbing wall substantially allows to absorb the transmitted beam passing through the beam splitter in order to reduce the phenomena of reflection and loss of contrast.

Advantageously, the light-absorbing wall is inclined at 45° and is specularly opposed with respect to the inclination of the first and second optical elements.

Preferably, a diaphragm having a hole is comprised between the first optical element and the second optical element. The diaphragm allows to collimate the first beam reflected by the mirror and directed toward the beam splitter.

In a preferred embodiment, the coupling means have the shape of a cover attachable to the mobile device integrating said optical accessory.

Preferably, a first polarizing filter disposed between the diaphragm and the second optical element, and a second polarizing filter disposed in front of the camera lens, are provided. The polarizing filters allow to reduce the light reflex on the cornea of the eye, most of all when the patient has miotic pupils.

In a second embodiment, said first and second optical elements have the shape of an optical guide element comprising at least one first face identifying said first reflective surface, and at least one second face identifying said partially reflective second surface.

In particular, said optical guide element is a monoblock made of a transparent material, wherein said first face has a polished surface and said at least one second face has a satin-finished surface which allows to generate said second light beam.

In detail, in this second embodiment, the source light beam strikes the first face of the optical guide element thereby generating the first reflected ray. The first reflected ray propagates into the optical guide element being subjected—in turn—to a plurality of total reflections before impinging on the second face of the optical guide element and generating the second reflected beam. The structure itself of the optical guide element allows to have total reflections of the first reflected ray. Therefore, in this arrangement, the term “first reflected ray” means the ray generated by the reflection with the first face, which is subjected to further inner reflections propagating into the optical element, until it impinges on the partially reflective second face of the same.

In particular, said first face is inclined at an angle φ comprised between 30° and 60° with respect to the lying plane (P).

In particular, said second face is inclined at an angle γ comprised between 30° and 60° with respect to the lying plane (P).

Preferably, the first and second faces of the optical guide element are inclined oppositely relative to one another. The optical guide element has a substantially trapezoidal longitudinal section.

In a third embodiment of the present invention, said first optical element is an optical guide element comprising at least one first face identifying said first reflective surface and a second face opposite to the first face. The second face has a transparent surface. On the contrary, the second optical element is a beam splitter positioned in correspondence with the second face of said optical guide element, such that the generated second reflected beam is directed coaxially with the axis of the eye to be observed.

In this modification, the first and second faces of the second optical guide element are inclined in the same direction, in particular the first and second faces are parallel to each other.

More particularly, the first reflected beam outgoing from the optical guide element, and specifically the second face, is substantially directed in the opposite direction with respect to the eye to be observed. The first reflected beam impinges on the beam splitter for generating the second reflected beam directed toward the eye to be observed. By positioning the beam splitter substantially in front of the lens, a second reflected beam in-line with the eye and the lens can be generated. This leads to the advantage of reducing the corneal reflex of the eye itself on the lens.

In a fourth embodiment of the invention, said first optical element is an optical fibre and said second optical element is a reflective element, in particular a prism or a mirror.

The reflective element, on which the first light beam outgoing from the optical fibre is incident, generates the second light beam directed in the optical field of the lens, illuminating the eye to be observed.

Preferably, at the exit of the optical fibre, a first polarizing filter for said first light beam is provided.

The second generated light beam, which strikes the eye to be observed, is inclined with respect to the optical axis. Advantageously, in the visual field of the lens, a second polarizing filter is provided, allowing to reduce the corneal reflex of the eye itself.

In a fifth embodiment, said first optical element is an optical fibre and said second optical element comprise said reflective element in turn associated to a beam splitter, such that the second light beam is reflected coaxially with the axis of the eye to be observed.

Moreover, in this way, only the normal polarization reaches the user eye, so that the corneal reflex of the eye itself is reduced.

Preferably, also in this modification, the first polarizing filter is provided at the exit of the optical fibre.

According to another aspect of the invention an optical device comprising an optical accessory according to claims 1 to 14, functionally associated to a mobile device, such as a smartphone or tablet having a camera lens and a light source, is provided.

### BRIEF LIST OF THE FIGURES

More features and advantages of the invention will be better understood by considering the following specification of several, but not exclusive, preferred embodiments, illustrated by way of example only and without limitation, with the support of the accompanying drawings, in which:

FIG. 1 is a perspective view of an optical accessory according to the present invention, applied to a mobile device, as a smartphone or tablet;

FIG. 2 is a schematic view of the optical accessory of FIG. 1;

FIG. 3 is a perspective view of the optical accessory of FIG. 1;

FIG. 4 shows a schematic view of a second embodiment of the optical accessory according to the present invention;

FIG. 5 shows a third embodiment of the optical accessory according to the invention;

FIG. 6 shows a schematic view of a fourth embodiment according to the present invention;

FIG. 7 shows a schematic view of a fifth embodiment still according to the present invention.

### DETAILED DESCRIPTION OF THE INVENTION

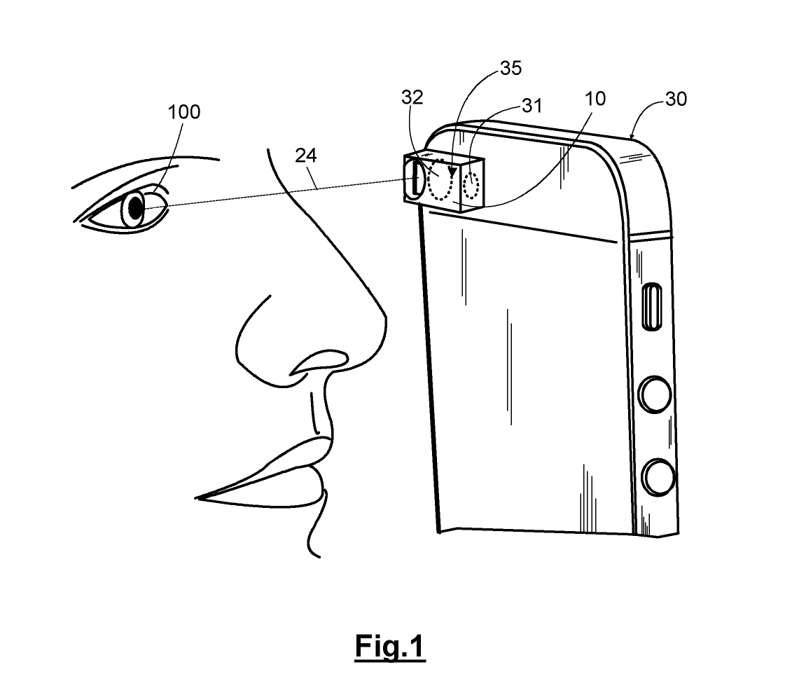

Referring to FIG. 1, an optical accessory 10 applied to a mobile device 30, as a smartphone or tablet, is shown. The mobile device 30 comprises a camera 35 provided with a camera lens 32 and a light source 31, for example a LED source.

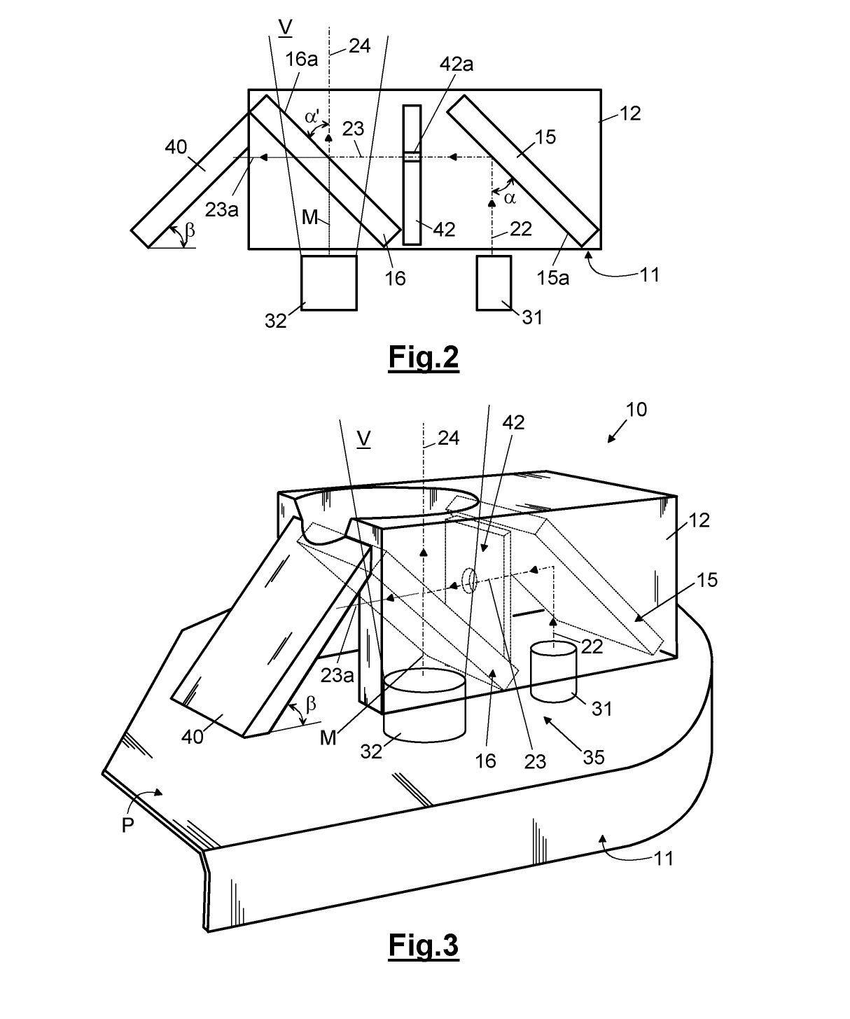

As better schematically shown in FIGS. 2 and 3, the optical accessory 10 is provided with coupling means 11 shaped so as to connect and position it in correspondence with the lens 32 and the light source 31 of the mobile device 30. For example, the coupling means 11 can be formed by a simple adhesive surface or a cover 11 (FIG. 3) to be snap coupled to the mobile device 30.

More particularly, the optical accessory 10 comprises a substantially box-shaped frame 12 in which a first optical element 15 provided with a first reflective surface 15a, in particular a mirror, is positioned. The surface 15a of the mirror 15 allows to reflect a source light beam 22 emitted by the light source 31. The total reflection of the source beam 22 generates a first reflected beam 23 directed toward the visual field V of the camera lens 32. In other words, the mirror 15 reflects the light beam in an area in front of the lens 32.

The accessory 10 further comprises a second optical element, in particular a beam splitter 16 provided with a partially—reflective second surface 16a. The first reflected beam 23 is incident on the surface 16a of the beam splitter 16 and it generates a second reflected beam 24 and a transmitted beam 23a.

The second surface 16a is oriented such as to be substantially parallel to the first surface 15a of the mirror 15. Both the surface 15a of the mirror 15 and the surface 16a of the beam splitter 16 are preferably flat surfaces.

The so-generated second beam 24 outgoing from the beam splitter 16 is directed to illuminate the patient eye 100 (FIG. 1) to be observed and is substantially inside the visual field V of the camera lens 32 and, in particular, is aligned to an axis or centerline M of the latter.

By positioning the optical accessory 10, associated to the mobile device 30, close to the eye 100 so that the light beam 24 is substantially on its optical axis, it is possible to carry out the examination of the eyeground or “fundus oculi” of the eye 100; at the same time, the optical accessory is simple, functional, portable and compact. With the optical accessory 10 assembled to the mobile device 30, in fact, the examination can be carried out very simply even through miotic pupils.

According to structural details, still referring to FIGS. 2 and 3, the surface 15a of the mirror 15 is inclined at an angle α of about 45°, in particular of about 52.5°, with respect to the lying plane P (FIG. 3) of the lens 32 and the light source 31, or, with respect to the source light beam 22, as shown in FIG. 2. The lying plane P is substantially the outer flat surface of the mobile device 30. The beam splitter 16 parallel to the mirror 15 is also inclined at an angle α′ of about 45°, in particular of 52.5°. In this way, the source light beam 22 is reflected by 90°, in particular by 97.5°, thereby generating the first reflected beam 23. Similarly the first beam 23, when it strikes the surface 16a of the beam splitter 16, is reflected by 90° and generates the second reflected beam 24 which strikes the eye 100.

The range of inclination of the α, α′ angles relative to the surfaces of the mirror 15 and the beam splitter 16 can change and being comprised preferably between 30° and 60°.

Advantageously, as above said, once the optical accessory 10 is positioned in line with the eye 100, the beam splitter 16 in front of the camera lens 32, i.e. in line with the centerline M of the visual field V, allows to carry out the examination of the eyeground and capture an image thereof by the lens 32.

In a preferred embodiment, the optical accessory 10 further comprises a light-absorbing wall 40 inclined at an angle β comprised between 30° and 60°, preferably of 45°. The inclination of the wall 40 is specularly opposed with respect to the inclination of the beam splitter 16 and the mirror 15. The light-absorbing wall 40 is placed opposite with respect to the mirror 15 and substantially allows to absorb the transmitted beam 23a passing through the beam splitter 16 and reduce the phenomena of reflection and loss of contrast of the lens 32.

Further, the accessory provides a diaphragm 42 provided with a calibrated hole 42a interposed between the mirror 15 and the beam splitter 16 on the path of the first reflected beam 23. The diaphragm 42 allows to substantially collimate the first reflected beam 23 and reduce its dispersion.

Still advantageously, in a way not shown in detail, the optical accessory 10 comprises a first polarizing filter disposed between the diaphragm 42 and the beam splitter 16 and a second polarizing filter disposed in front of the camera lens 32. These are advantageously employed to reduce the phenomena of light reflection on the cornea of the eye 100, especially in patients with miotic pupils.

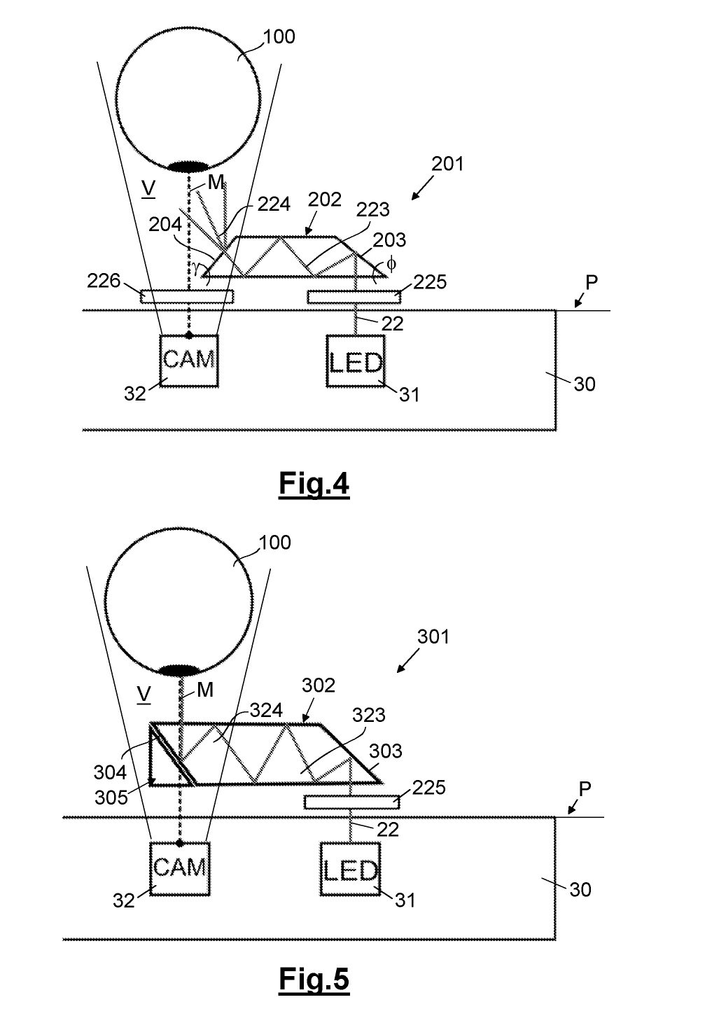

Referring to FIG. 4, a second embodiment of the optical accessory is shown, generally referred to with the numeral 201, wherein parts structurally and functionally corresponding to the optical accessory 10, which is illustrated with reference to FIGS. 1 to 3, maintain the already used numerals.

In this second embodiment, the first and the second optical elements have the shape of an optical guide element 202 comprising at least one first face 203 identifying a first reflective surface, and at least one second face 204 identifying a partially reflective second surface.

In particular, the optical guide element 202 is a monoblock made of a transparent material, in which the first face 203 has a polished surface whereas the second face 204 has a satin-finished surface, that allows to generate a second substantially-divergent light beam 224 directed toward the eye 100 to be observed, always inside the visual field V of the lens.

In more details, the optical guide element is made of a transparent and homogeneous material, for example plastic (PMMA, polycarbonate, etc.) or glass.

According to the operation principle, the source light beam 22 strikes the first face 203 of the optical guide element 202 thereby generating the first reflected ray 223. The first reflected ray 223 propagates in the optical guide element 202 being subjected—in turn—to a plurality of total reflections before impinging on the second face 204 of the partially-reflective optical guide element and generating the second reflected beam 224. The structure itself of the optical guide element 202 allows to have total reflections of the first reflected ray 223.

Structurally, the first face 203 is inclined at an angle φ comprised between 30° and 60° with respect to the lying plane P, while the second face 204, being oriented oppositely with respect to the first face 203, preferably is inclined at an angle γ comprised between 30° and 60° with respect to the lying plane P.

In a third embodiment of the accessory, generally referred to as 301 and shown in FIG. 5, the first optical element is an optical guide element 302 structurally shaped as the optical guide element 202 of FIG. 4, which comprises at least one first face 303 identifying the first reflective surface and a substantially transparent second face 304. On the contrary, the second optical element is a beam splitter 305 positioned in correspondence with the second face 304. By positioning the beam splitter 305, a second reflected beam 324 can be generated and directed coaxially with the axis of the eye M to be observed.

Structurally, the first face 303 and second face 304 of the optical guide element 302 are concordantly inclined on the same side and, in particular, are substantially parallel to each other. The first reflected beam 323 outgoing from the optical guide element 302 is directed substantially in the opposite direction with respect to the eye 100 to be observed. This beam reflects on the beam splitter 305 thereby generating the second reflected beam 324 directed toward the eye 100. The second reflected beam 324 is coaxial with the axis M of the eye and with the lens 32 in the visual field V, thereby leading to the advantage of reducing the corneal reflex of the eye itself on the lens.

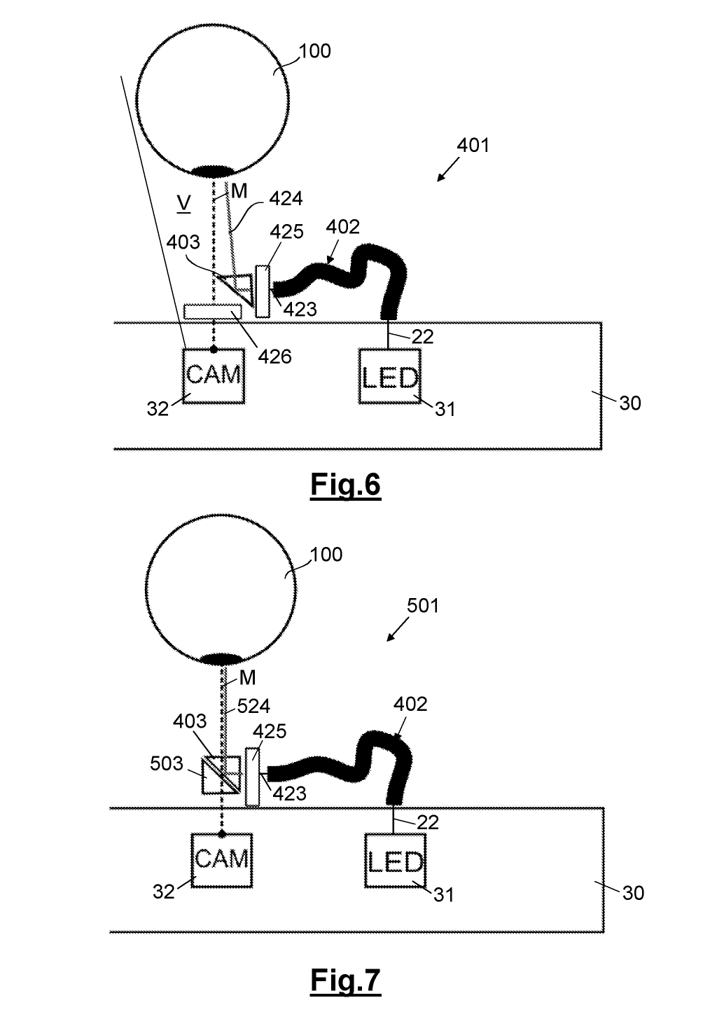

In a fourth embodiment with reference to FIG. 6, an optical accessory generally referred to as 401 is shown, providing an optical fibre 402 which is the first optical element, and a reflective element 403, particularly a prism, which is the second optical element. Reflective element means an at least partially-reflective element used to orient and split the light beam.

The reflective element 403, on which the first light beam 423 outgoing from the optical fibre 402 is incident, generates the second light beam 424 directed in the optical field V of the lens 32, which illuminates the eye 100 to be observed.

The second light beam 424 is inclined with respect to the optical axis M.

In this modification, at the exit of the optical fibre 402 a first polarizing filter 425 of the first light beam 423 is provided, as well as a second polarizing filter 426 positioned in front of the lens 32 for reducing the phenomena of corneal reflection of the eye 100 itself.

In a fifth embodiment shown in FIG. 7, the optical accessory, generally referred to as 501, comprises the optical fibre 402, the reflective element 403 and additionally a beam splitter 503 associated to said reflective element 403. The beam splitter 503, functionally combined with the reflective element 403, allows to reflect the second light beam 524 coaxially with the axis M of the eye 100 thereby transmitting to the user eye only the normal polarization in order to reduce or cancel the corneal reflex.

1. An optical accessory of a mobile device, comprising:
coupling means for the detachable association with said mobile device, wherein said coupling means allow the positioning of said optical accessory in correspondence with both a camera lens and a light source provided by said mobile device, wherein said optical accessory further comprises:
a first optical element having a first reflective surface apt to reflect a source light beam emitted by said light source and to generate a first reflected beam substantially directed toward a visual field of said camera lens;
a second optical element having a partially reflective second surface on which said first reflected beam is incident and apt to generate a second reflected beam directed toward an eye to be examined inside the visual field of said camera lens in such a way to allow the view of the eyeground of said eye.

2. An optical accessory according to claim 1, wherein the first optical element is a mirror and said second optical element is an optical beam splitter.

3. An optical accessory according to claim 1, wherein said first surface and said partially reflective second surface are parallel to each other.

4. An optical accessory according to claim 1, wherein said first surface of the first optical element is inclined at an angle comprised between 30° and 60° with respect to a plane in which said camera lens and said light source lie, and wherein said second surface of the second optical element is inclined at an angle comprised between 30° and 60° with respect to said plane.

5. An optical accessory according to claim 1, further comprising a light-absorbing wall which is associated to the second optical element and placed opposite to the first optical element.

6. An optical accessory according to claim 5, wherein said light-absorbing wall is inclined at an angle comprised between 30° and 60°, preferably at 45°, with respect to the lying plane, and which is specularly opposed with respect to the inclination of both respective surfaces of said first optical element and said second optical element.

7. An optical accessory according to claim 1, wherein a diaphragm having a hole is comprised between said first optical element and said second optical element.

8. An optical accessory according to claim 1, wherein said coupling means have the shape of a cover attachable to said mobile device.

9. An optical accessory according to claim 7, further comprising a first polarizing filter disposed between said diaphragm and said second optical element, and a second polarizing filter disposed in front of said camera lens.

10. An optical accessory according to claim 1, wherein said first and second optical elements have the shape of an optical guide element which comprises at least one first face identifying said first reflective surface, and at least one second face identifying said partially reflective second surface, wherein said optical guide element is a monoblock made of a transparent material, wherein at least said first face has a polished surface, and at least said second face has a satin-finished surface, which allows to generate a second light beam directed toward the eye to be observed in the visual field of said lens.

11. An optical accessory according to claim 1, wherein said first optical element is an optical guide element which comprises at least one first face identifying said first reflective surface apt to generate the first reflected beam, and a second substantially transparent face opposite to said first face, wherein said optical guide element is a monoblock made of a transparent material, and wherein said second optical element is a beam splitter positioned in correspondence with said second face, so that when the first reflected beam impinges on said beam splitter, the second generated reflected beam is directed coaxially with the axis of the eye to be observed in the visual field of said lens.

12. An optical accessory according to claim 1, wherein said first optical element is an optical fibre and said second optical element is a reflective element, in particular a prism., wherein said reflective element, on which the first light beam coming out of the optical fibre is incident, generates the second light beam directed toward the eye to be observed in the optical field of said lens.

13. An optical accessory according to claim 12, wherein a beam splitter associated to said reflective element is provided such that the second generated light beam is coaxial with the axis of the eye to be observed in the visual field of said lens.

14. An optical accessory according to claim 1, wherein a first polarizing filter is disposed at the exit of said first optical element and in combination with a second polarizing filter positioned in the visual field of said lens.

15. An optical device comprising an optical accessory according to claim 1, functionally associated to a mobile device, such as a smartphone or tablet having a camera lens and a light source.
